(19) United States
(12) Patent Application Publication
(10) Pub. No.: US 2021/0333283 A1
(43) Pub. Date: Oct. 28, 2021

(54) CANCER-RELATED ACTIVITY SENSORS

(71) Applicant: GLYMPSE BIO, INC., Cambridge, MA (US)

(72) Inventors: James Bowen, Cambridge, MA (US); Faycal Touti, Cambridge, MA (US)

(21) Appl. No.: 17/238,585

(22) Filed: Apr. 23, 2021

Related U.S. Application Data
(60) Provisional application No. 63/015,340, filed on Apr. 24, 2020.

(51) Int. Cl. G01N 33/58

(52) U.S. Cl. CPC G01N 33/58; G01N 2333/95; G01N 2800/52; G01N 2800/56

(57) ABSTRACT

An activity sensor sensitive to enzymes indicative of the presence of cancer is used to provide non-invasive reporting of tumor development and response to anticancer therapies useful in determining suitability and effectiveness of various treatments including immuno-oncological therapies. Localized reporters are used in patient stratification in clinical trials, monitoring of drug response or disease progression, and differentiating between anti-tumor immune response, tumor progression, and off-target immune response. Activity sensors may include tuning domains to modulate tissue localization and residency. Periodic measurements of activity sensor reporters may be analyzed to determine a velocity value indicative of disease prognosis.

### TECHNICAL FIELD

The invention relates to activity sensors for cancer diagnosis and treatment.

### BACKGROUND

Despite enormous money and effort expended on research and treatment, cancer continues to cause suffering and death across large portions of the population. Over 1.7 million new cases of cancer and over 600,000 cancer deaths are expected to occur in the US in 2019. Cancer immunotherapy or immuno-oncology (I-O) is a recently developed field that has shown promise in treating various forms of cancer. I-O refers to the use of a patient's own immune system to attack their cancer. I-O is a broad category and includes passive as well as active techniques. Passive techniques involve the augmentation of a patient's existing anti-tumor immune response through, for example, immune checkpoint inhibitors (e.g., CTLA-4 blockade, PD-1 inhibitors, or PD-L1 inhibitors) that can disrupt tumor defenses against immune system attacks. Active techniques include targeted immune therapies such as engineered CAR-T cells programmed to target tumor-specific antigens.

Unfortunately, gaining insight into the tumor environment without invasive biopsies or potentially misleading imaging is difficult. Successful responses to immunotherapy can resemble disease progression when imaged and the mechanisms of effective responses are poorly understood. Lack of specific biomarkers and detailed disease information present an obstacle to clinical trials, I-O therapy development, and personalized treatments.

### SUMMARY

The invention provides non-invasive monitoring and/or detection of characteristics of cancer progression, localized immune system activity, and immuno-therapeutic response. According to the invention, activity monitors comprise reporters coupled to a carrier, such that the activity monitor is released for detection in response to a therapeutic condition. Activity monitors of the invention are useful for determining disease state, monitoring progression of disease, predicting and monitoring recurrence, assessing therapeutic response and predicting therapeutic efficacy. For example, using tumor-localized activity sensors with cleavable reporters sensitive to immunological enzymes and enzymes indicative of cell death allows for rapid prediction of therapeutic response and detailed monitoring of cancer progression and evolution. Accordingly, unsuccessful therapies can quickly be identified as such and development of resistances to any given oncotherapy can be flagged resulting in more responsive personalized medicine with less wasted time and less opportunity for tumor progression. Systems and method of the invention can include activity sensors with cleavable linkers sensitive to proteases differentially expressed in immune responses including inflammation and apoptosis. Activity sensors sensitive to proteases associated with necrotic cell death associated with natural tumor progression can also be included to provide additional information on cancer progression. Comparison of inflammation/apoptosis-related protease levels to necrosis-related protease levels can provide a more detailed view of cancer progression and I-O treatment response. Sensors of the invention are also useful to map biodistribution. Thus, it is possible to identify the distribution of cancer cells and the metastatic state of disease using constructs of the invention.

Caspases (cysteine-aspartic proteases, cysteine aspartases or cysteine-dependent aspartate-directed proteases) are associated with programmed cell death (e.g., apoptosis) and inflammation and, therefore, activity sensors engineered with caspase-cleavable reporters as described herein can provide a synthetic biomarker indicative of immune response. Other proteases indicative of an immune response include serine proteases such as granzymes, neutrophil elastase, cathepsin G, proteinase 3, chymase, and tryptase. Certain proteases are also associated with cancer progression and tumor growth. Tumor-associated proteases may be differentially expressed depending on the type of cancer (e.g., tumor-associated differentially expressed gene-14 (TADG-14) is highly overexpressed in ovarian cancer). As such, cancer-related activity sensors can provide diagnostic and prognostic information for a variety of cancers based on the reported levels of key enzymes. Cancer-related activity sensors may be sensitive to any tumor-related protease including those described in Vasiljeva, et al., 2019, The multifaceted roles of tumor-associated proteases and harnessing their activity for prodrug activation, Biol Chem., 400(8), pp. 965-977, and Dudani, et al., 2018, Harnessing protease activity to improve cancer care, Annu. Rev. Cancer Biol., 2:353-76, the contents of each of which are incorporated herein by reference.

As discussed below, the cancer-related synthetic biomarkers can include tuning domains to localize the activity sensors to tumors in order to provide a tumor-specific picture of immune response useful in differentiating cancer-specific therapeutic effects from systemic or off-target immune responses.

Activity sensors can include a molecular carrier structure linked to one or more detectable analytes via cleavable linkers. The presence and amount of immunological enzymes as measured by activity sensor reporter levels in a patient sample can be used to determine innate or augmented immuno-oncological responses in a patient. For example, a baseline signal of caspase and serine protease reporters from tumor-localized activity sensors can indicate a non-responsive tumor when measured after treatment or an immunologically-cold tumor when measured before treatment. An indication of an immunologically-cold tumor can indicate that the patient is not likely to respond to checkpoint inhibitors. Slightly elevated signals of caspase and serine protease reporters from tumor-localized activity sensors measured pre-treatment can be indicative of an immunologically-hot tumor where the patient may be a good candidate for checkpoint inhibitors. A high signal of caspase and serine protease reporters from tumor-localized activity sensors measured during or after treatment may be indicative of a desired immuno-oncological therapeutic response.

Reported levels of necrosis-related proteases such as calpain and cathepsin can provide information regarding necrotic cell death to supplement the immuno-oncological information and help differentiate between tumor progression and pseudoprogression.

As noted, activity sensors and methods of the invention can be applied to I-O treatments to predict or observe I-O drug responses in patients. By providing more detailed and relevant information regarding individual patients, new patterns may be identified among responders and non-responders in trials and the information obtained via the activity sensors can be used for better patient stratification during clinical trials and may help identify patient subpopulations that stand to benefit from specific treatments. Accordingly, activity sensors and methods of the invention may support the clearance of helpful therapies that would have previously been discarded based on limited understanding of patient characteristics relevant to responsiveness or adverse effects.

Activity sensors act as synthetic biomarkers that can be programmed to provide non-invasive reporting of any enzyme level in a specific target tissue through engineering of an enzyme-specific cleavage site in the activity sensor. For example, the activity sensors may be a multi-arm polyethylene glycol (PEG) scaffold linked to four or more polypeptide reporters as the cleavable analytes. The cleavable linkers are specific for different enzymes whose activity is characteristic of a condition to be monitored (e.g., a certain stage or progression in cancerous tissue or an immune response). When administered to a patient, the activity sensors locate to a target tissue, where they are cleaved by the enzymes to release the detectable analytes. The analytes are detected in a patient sample such as urine. The detected analytes serve as a report of which enzymes are active in the tissue and, therefore, the associated condition or activity.

Because enzymes are differentially expressed under the physiological state of interest such as a repressed immune response, an active immune response, or tumor expansion or reduction, analysis of the sample provides a non-invasive test useful in assessing or predicting a patient's response to various I-O therapies. Because the activity sensors provide a non-invasive snapshot of the tumor microenvironment, frequent monitoring becomes practical. Access to up-to-date information on disease progression and therapeutic response can allow for quicker decisions for assessing safety and efficacy and are particularly useful in monitoring immune resistances as they arise.

In certain embodiments, activity sensors and methods can be used to distinguish between general immune responses, tumor-specific immune-responses, and tumor progression. For example, the phenomenon of pseudoprogression refers to the apparent progression of a tumor under radiographic imaging while the size increase is in fact caused by swelling in response to a successful immune response. That misinterpretation can lead to the abandonment of a successful treatment. By providing detailed information on immune system activity in the cancerous tissue, activity sensors of the invention can prevent such misinterpretations and compliment traditional monitoring methods.

In other instances, a general immune response (e.g., due to a viral infection) can be misinterpreted as a true anti-tumor immune response. Activity sensors of the invention can be localized to a specific tissue including a target tumor through the use of tuning domains to increase uptake in the target environment. Accordingly, any observed activity reporters in a patient sample can be safely attributed to the target environment as opposed to an unrelated, off—target immune response.

Additionally, the information provided using the activity sensors can be used to distinguish hot tumors from cold tumors. Immunologically cold tumors refer to those tumors with few infiltrating T cells that do not provoke a strong response by the immune system. Hot tumors, in contrast, contain high levels of infiltrating T cells and more antigens and are more likely to trigger a strong immune response. Accordingly, a hot tumor, already recognized by the patient's immune system as a target, may be a good candidate for passive treatments such as a checkpoint inhibitor to simply augment the patient's existing immune response.

As noted herein, activity sensors may include a variety of different cleavable reporters sensitive to different enzymes. Furthermore many different activity sensors can be administered and analyzed simultaneously. The reporter molecules can be distinguishable from one another such that multiplex analysis of a variety of protease activities can be accomplished, painting a more detailed picture of the tumor microenvironment than previously possible using natural biomarkers. Reporters may include dyes, such as a near-IR dye, a nucleic acid or protein barcode, and others.

In certain embodiments, I-O activity sensors, acting as synthetic biomarkers, may be administered and measured periodically to provide a chronological mapping of various enzyme levels. In addition to point-in-time information, the rate of change in measurements of the various enzyme levels can be examined to provide velocity information. Such a panel is useful for providing an indication of health, which is applicable even to healthy individuals and provides another data point beyond traditional longitudinal monitoring for disease progression and therapeutic response.

In various embodiments the activity sensor carrier structure can include multiple molecular subunits and may be, for example, a multi-arm polyethylene glycol (PEG) polymer, a lipid nanoparticle, or a dendrimer. The detectable analytes may be, for example, polypeptides that are cleaved by proteases that are differentially expressed in tissue or organs experiencing an immune response or undergoing a disease progression. Because the carrier structure and the detectable analytes are biocompatible molecular structures that locate to a target tissue and are cleaved by disease or immune-response-associated enzymes to release analytes detectable in a sample, compositions of the disclosure provide non-invasive methods for detecting and characterizing the state of an organ or tissue. Because the compositions provide substrates that are released as detectable analytes by enzymatic activity, quantitative detection of the analytes in the sample provide a measure of rate of activity of the enzymes in the organ or tissue. Thus, methods and compositions of the disclosure provide non-invasive techniques for assessing both the stage and the rate of progression of cancer or response to I-O therapies.

Activity sensors may take the form of cyclic peptides that are naturally resistant to off-target degradation. The target environment may be a tumor microenvironment in which a specific enzyme or set of enzymes are differentially-expressed. A cyclic peptide may be engineered with cleavage sites specific to enzymes in the tumor (e.g., unique enzymes expressed preferentially in the tumor). The engineered peptide, in its cyclic form, can travel through the blood and other potentially harsh environments protected against degradation by common non-specific proteases and without interacting in a meaningful way with off-target tissues. Only upon arrival within the specific target tissue and exposure to the required enzyme or combination of enzymes, the cyclic peptide is cleaved to produce a linear molecule that is capable of clearance and sample observation. For purposes of the application and as will be apparent upon consideration of the detailed description thereof, a linear peptide is any peptide that is not cyclic. Thus, for example, a linearized peptide may have various branch chains.

Cyclic peptides can be engineered with other cleavable linkages, such as ester bonds in the form of cyclic depsipeptides in which the degradation of the ester bond releases a linearized peptide ready to react with its target environment. Thioesters and other tunable bonds can be included in the cyclic peptide to create a timed-release in plasma or other environments. See Lin and Anseth, 2013 Biomaterials Science (Third Edition), pages 716-728, incorporated herein by reference.

Macrocyclic peptides may contain two or more protease-specific cleavage sequences and can require two or more protease-dependent hydrolytic events to release a reporter peptide or a bioactive compound. The protease-specific sequences can be different in various embodiments. In cases where cleavage of multiple sites is required to release the linearized peptide, different protease-specific sequences can increase specificity for the release as the presence of at least two different target-specific enzymes will be required. The specific and non-specific proteolysis susceptibility and rate can be tuned through manipulation of peptide sequence content, length, and cyclization chemistry.

Activity sensors may include additional molecular structures to influence trafficking of the peptides within the body, or timing of the enzymatic cleavage or other metabolic degradation of the particles. The molecular structures may function as tuning domains, additional molecular subunits or linkers that are acted upon by the body to locate the activity sensor to the target tissue under controlled timing. For example, the tuning domain may target the particle to specific tissue or cell types. Trafficking may be influenced by the addition of molecular structures in the carrier polymer by, for example, increasing the size of a PEG scaffold to slow degradation in the body.

In certain embodiments, the invention provides a tunable activity sensor that reveals enzymatic activity associated with a physiological state, such as disease. When the activity reporter is administered to a patient, it is trafficked through the body to specific cells or specific tissues. For example, in a patient with lung cancer, the activity sensor may be tuned to localize in the cancerous tissue through, for example, the use of tuning domains preferentially trafficked to lung tissue or tumor tissue. The activity sensors can include cleavable reporter molecules sensitive to enzymes indicative to an immune response or a stage of tumor progression or regression. Subsequent observation and/or tracking of reporter levels in a patient sample (e.g., urine) will then provide an indication of the progression and/or therapeutic response of the patient's lung cancer.

The sensor may be designed or tuned so that it remains in circulation, e.g., in blood, or lymph, or both. If enzymes that are differentially expressed under conditions of a particular disease are present, those enzymes cleave the reporter and release a detectable analyte. Cyclic peptide activity sensors may be used to resist non-specific degradation of the peptide in circulation while still providing an accessible substrate for cleavage by the target proteases.

Aspects of the invention include methods of monitoring cancer progression including administering to a patient suspected of having cancer an activity sensor comprising a carrier linked to a reporter molecule by a cleavable linker containing the cleavage site of an enzyme indicative of a characteristic of a tumor environment. A sample such as a urine sample can be collected from the patient and analyzed to detect the presence or lack of the reporter, where presence of the reporter is indicative of the characteristic.

The characteristic may be an active immune response and the patient is undergoing immuno-oncological treatment, wherein presence of the reporter is indicative of therapeutic effect of the immuno-oncological treatment. The presence of the reporter may also be indicative of other treatment modalities, such as, for example, chemotherapy, targeted therapies, or radiation therapy. The activity sensor may include a tuning domain operable to localize the activity sensor in a target tumor. The characteristic can be a checkpoint inhibited immune response and, wherein presence of the reporter is indicative of a predicted therapeutic response to a checkpoint inhibitor therapy. Methods may include stratifying the patient in a clinical trial based on the detection of the reporter in the sample.

In certain embodiments, the analyzing step may include quantifying a level of the reporter in the sample and the method can include periodically repeating the administering, collecting, and analyzing steps to prepare a chronological series of reporter levels from which a velocity of the characteristic can be determined that is indicative of cancer progression in the patient.

In certain aspects the invention may include an activity sensor for monitoring cancer progression, the activity sensor including a carrier comprising one or more molecular subunits; a plurality of detectable reporters, each linked to the carrier by a cleavable linker containing the cleavage site of an enzyme indicative of a characteristic of a tumor environment; and a tuning domain operable to localize the activity sensor in a target tumor, wherein the activity sensor reports activity of one or more enzymes by releasing the reporters upon cleavage by the one or more enzymes. The characteristic may be an active immune response in a patient undergoing immuno-oncological treatment. In various embodiments, the characteristic can be a checkpoint inhibited immune response.

The tuning domain may include ligands for receptors of the target tumor. The carrier may include a polyethylene glycol (PEG) scaffold of covalently linked PEG subunits. In certain embodiments, the carrier includes a multi-arm PEG scaffold and the detectable reporters and cleavable linkers each comprise a polypeptide linked to the PEG scaffold. The ligands may each include a small molecule, a peptide, an antibody, a fragment of an antibody, a nucleic acid, or an aptamer. Activity sensors may include a plurality of reporters and a plurality of tuning domains, wherein the tuning domains comprise biocompatible polymer linked to the reporters. The activity sensor may include a cyclic peptide linearized upon cleavage of the cleavable linker.

### BRIEF DESCRIPTION OF THE DRAWINGS

FIG. 1 diagrams steps of a method for monitoring cancer progression.

FIG. 2 shows an activity sensor.

FIG. 3 shows an engineered macrocyclic peptide.

### DETAILED DESCRIPTION

The invention provides detailed information on cancer-related immune responses through the use of localized activity sensors to report on differential expression of immunologically associated enzymes. Such activity sensors can include a variety of reporter molecules that are detectable in a body fluid sample such as urine but are only released from the body upon contact with cleavage enzymes associated with localized immune responses or cancer progression. Accordingly, detection of the reporters in the sample is indicative of the differential expression of the enzymes in the target tissue and the presence of the associated immune activity (e.g., immuno-therapeutic response or an immunologically-hot tumor). By preferentially targeting cancerous tissues and engineering cleavage sites specific to enzymes differentially expressed under various conditions, activity sensors of the invention can provide insight into cancer progression and predicted or actual immuno-therapeutic responses not possible with existing imaging or systemic monitoring techniques.

Several proteases are known to be associated with inflammation and programmed cell death (e.g., including apoptosis, pyroptosis and necroptosis). The localized levels of those proteases are accordingly indicative of immune system activity. Caspases (cysteine-aspartic proteases, cysteine aspartases or cysteine-dependent aspartate-directed proteases) are a family of protease enzymes including a cysteine in their active site that nucleophilically cleaves a target protein only after an aspartic acid residue. Caspase-1, Caspase-4, Caspase-5 and Caspase-11 are associated with inflammation. Serine proteases also function in apoptosis and inflammation and their differential expression is therefore also indicative of an immune response. Immune cells express serine proteases such as granzymes, neutrophil elastase, cathepsin G, proteinase 3, chymase, and tryptase.

In various embodiments, it may be useful to differentiate between programmed cell death indicative of an immune response and necrosis naturally found during tumor progression. In contrast to programmed cell death, where caspases and serine proteases are the primary proteases, calpains and lysosomal proteases (e.g., cathepsins B and D) are the key proteases in necrosis. Accordingly, calpain and cathepsin levels indicated by activity sensor reporter measurements can provide information regarding necrotic cell death to supplement the immuno-oncological information.

Activity sensors and methods of the invention can be applied to I-O treatments to observe I-O drug responses in patients. For example, activity sensors with cleavage sites sensitive to caspases, serine proteases, calpains, and cathepsins can be administered during or after I-O treatment and reporter levels in patient samples can be used to monitor therapeutic response. A baseline signal of caspases or serine proteases in patient samples is indicative of a non-responsive tumor. The baseline level can be determined experimentally through data collected from patient populations or pre-treatment data from the patient undergoing treatment. Increased signals of caspases and serine proteases during or after treatment relative to a baseline level can be indicative of a desired immuno-oncological response. Tracking the levels of calpain or cathepsin signals can provide additional information on non-immunological cell death that may be associated with tumor progression.

The depth of information provided from activity sensors regarding tumor characteristics and patient response to treatments can offer new factors for use in patient stratification for clinical trials, for example. Stratification is the partitioning of subjects and results by a factor other than the treatment given. Stratification is traditionally done by factors such as gender, age, or other demographic details but the addition of detailed patient and tumor information obtained via activity sensors of the invention can provide more practical and meaningful groups for stratification. Examining patient responses in view of such groupings can be used to eliminate variables to better interpret results and map adverse events or therapeutic efficacy to causative patient characteristics. The information obtained via the activity sensors can also be analyzed and combined with treatment results (positive or adverse) to use in future testing and identification of good candidates for treatment with cancers likely to respond while not experiencing adverse reactions.

Activity sensors act as synthetic biomarkers that can be programmed to provide non-invasive reporting of any enzyme level in a specific target tissue through engineering of an enzyme-specific cleavage site in the activity sensor. When administered to a patient, the activity sensors locate to a target tissue using, for example, target-specific tuning domains. Once localized, they are cleaved by the enzymes to release the detectable analytes. The analytes are detected in a patient sample such as a urine sample. The detected analytes serve as a report of which enzymes are active in the tissue and, therefore, the associated condition or activity. Localization allows activity sensors to report on the conditions of a target tissue without contamination of off-target information. That ability is useful in differentiating anti-tumor immune responses indicative of successful I-O treatment from an off-target immune response that may, for example, be occurring in response to a viral infection. For example a general increase in immunological enzymes (e.g., caspases or serine proteases) may result from a systemic or off-target immune response such as a viral infection. The ability of the invention to provide tumor-specific information regarding immune system activity avoids misinterpretation of a general immune response as a desired anti-tumor response.

Additionally, because activity sensor monitoring is non-invasive, frequent monitoring is more feasible and up-to-date information on disease progression and therapeutic response can be used for quicker decision making and safety and efficacy assessment. In the context of I-O treatments, frequent monitoring can be used to quickly identify resistances to treatment as they develop. For example, as cancers progress, they continue to mutate and neo-antigens used to target immunotherapies may no longer be expressed, causing therapeutic effectiveness to diminish. The ability to quickly identify such changes through changes in expression levels of immunological enzymes can lead to faster therapy changes, perhaps before significant cancer progression or recurrence.

The ability to monitor immune response in specific tissue (e.g., a tumor) can also prevent misinterpretation of observed tumor size increases known as pseudoprogression. Tracking anti-tumor immune response allows swelling due to desired anti-tumor immune responses to be distinguished from tumor growth due to disease progression. For example, radiographic imaging showing an increase in tumor size during I-O treatment would logically lead to the conclusion that the cancer is progressing and the therapy was not effective. However, the tumor may in fact be swelling to an inflammatory response indicative of a successful I-O treatment. That confusion may have historically led to the abandonment of an effective treatment. Using methods of the invention, tumor imaging and other indicators can be supplemented with tumor-specific immunological enzyme activity levels so that, an increased level of caspases or serine proteases accompanying increased tumor size can be correctly interpreted as a desired I-O therapeutic response.

An immunological enzyme may be an enzyme produced as part of an immune response. For example, an immunological enzyme may include an enzyme produced by immune cells.

In some embodiments, a cancer specific enzyme (but not an immune derived enzyme or one involved in cell death) may be indicative of a therapeutic effect. For example, in certain instances decreased activity of an enzyme associated with cancer growth or angiogenesis is indicative of the treatment response. A person of skill in the art will appreciate that activity sensors and methods of use described herein may be employed to determine a therapeutic response by measuring for one or more cancer specific enzymes using activity sensors described herein.

Activity sensors and methods of the invention can also be used to evaluate patient suitability for an I-O therapy. For example, activity sensors can report on enzymes differentially expressed in a patient's natural immune recognition and response to cancerous tissue. Such activity sensors can be administered before any I-O treatment in order to differentiate between hot tumors and cold tumors. Where a patient has a tumor that contains high levels of infiltrating T cells and more antigens, they may be a good candidate for passive treatments such as a checkpoint inhibitor to augment the patient's existing immune response. Checkpoint proteins include CTLA-4 (cytotoxic T lymphocyte associated protein 4), PD-1 (programmed cell death protein 1), and PD-L1 (programmed death ligand 1) are known to mask tumors from immune detection or response and various inhibitors for each are known. Where activity sensors sensitive to immune system recognition indicate a hot tumor, such checkpoint inhibitor therapies may be indicated. For example, a higher-than-baseline level of increased level of caspase or serine protease activity in a pre-treatment tumor can be observed using activity sensors as discussed herein and would indicate some immune system recognition and activity at the tumor site. The presence of an innate immune recognition and response supports a conclusion that cancer progression is reliant on checkpoint protein manipulation and the administration of a checkpoint inhibitor may prove effective for that patient.

Enzyme-specific reporters can be multiplexed on single activity sensors or in many different activity sensors that are administered and analyzed simultaneously. The reporter molecules can be specific for each enzyme such that they can be distinguished in multiplex analysis. In certain embodiments, I-O activity sensors, acting as synthetic biomarkers, may be administered and measured periodically to provide a chronological mapping of various enzyme levels. Studies have found that biomarker velocity (the rate of change in biomarker levels over time) may be a better indicator of disease progression (or regression) than any single threshold. The same principle can be applied to the activity sensors of the invention acting as synthetic biomarkers.

Activity sensors can include a carrier, at least one reporter linked to the carrier and at least one tuning domain that modifies a distribution or residence time of the activity sensor within a subject when administered to the subject. The activity sensor may be designed to detect and report enzymatic activity in the body, for example, enzymes that are differentially expressed during immune responses or during tumor progression or regression. Dysregulated proteases have important consequences in the progression of diseases such as cancer in that they may alter cell signaling, help drive cancer cell proliferation, invasion, angiogenesis, avoidance of apoptosis, and metastasis.

The activity sensor may be tuned via the tuning domains in numerous ways to facilitate detecting enzymatic activity within the body in specific cells or in a specific tissue. For example, the activity sensor may be tuned to promote distribution of the activity sensor to the specific tissue or to improve a residence time of the activity sensor in the subject or in the specific tissue. Tuning domains may include, for example, molecules localized in rapidly replicating cells to better target tumor tissue.

When administered to a subject, the activity sensor is trafficked through the body and may diffuse from the systemic circulation to a specific tissue, where the reporter may be cleaved via enzymes indicative of cancer progression or immune response. The detectable analyte may then diffuse back into circulation where it may pass renal filtration and be excreted into urine, whereby detection of the detectable analyte in the urine sample indicates enzymatic activity in the target tissue.

The carrier may be any suitable platform for trafficking the reporters through the body of a subject, when administered to the subject. The carrier may be any material or size suitable to serve as a carrier or platform. Preferably the carrier is biocompatible, non-toxic, and non-immunogenic and does not provoke an immune response in the body of the subject to which it is administered. The carrier may also function as a targeting means to target the activity sensor to a tissue, cell or molecule. In some embodiments the carrier domain is a particle such as a polymer scaffold. The carrier may, for example, result in passive targeting to tumors or other specific tissues by circulation. Other types of carriers include, for example, compounds that facilitate active targeting to tissue, cells or molecules. Examples of carriers include, but are not limited to, nanoparticles such as iron oxide or gold nanoparticles, aptamers, peptides, proteins, nucleic acids, polysaccharides, polymers, antibodies or antibody fragments and small molecules.

The carrier may include a variety of materials such as iron, ceramic, metallic, natural polymer materials such as hyaluronic acid, synthetic polymer materials such as poly-glycerol sebacate, and non-polymer materials, or combinations thereof. The carrier may be composed in whole or in part of polymers or non-polymer materials, such as alumina, calcium carbonate, calcium sulfate, calcium phosphosilicate, sodium phosphate, calcium aluminate, and silicates. Polymers include, but are not limited to: polyamides, polycarbonates, polyalkylenes, polyalkylene glycols, polyalkylene oxides, cellulose ethers, cellulose esters, nitro celluloses, polymers of acrylic and methacrylic esters, methyl cellulose, ethyl cellulose, and hydroxypropyl cellulose. Examples of non-biodegradable polymers include ethylene vinyl acetate, poly(meth) acrylic acid, polyamides, copolymers and mixtures thereof.

Examples of biodegradable polymers include synthetic polymers such as polymers of lactic acid and glycolic acid, poly-anhydrides, polyurethanes, and natural polymers such as alginate and other polysaccharides including dextran and cellulose, collagen, albumin and other proteins, copolymers and mixtures thereof. In general, these biodegradable polymers degrade either by enzymatic hydrolysis or exposure to water in vivo, by surface or bulk erosion. These biodegradable polymers may be used alone, as physical mixtures (blends), or as co-polymers.

In preferred embodiments, the carrier includes biodegradable polymers so that whether the reporter is cleaved from the carrier, the carrier will be degraded in the body. By providing a biodegradable carrier, accumulation and any associated immune response or unintended effects of intact activity sensors remaining in the body may be minimized.

Other biocompatible polymers include PEG, PVA and PVP, which are all commercially available. PVP is a non ionogenic, hydrophilic polymer having a mean molecular weight ranging from approximately 10,000 to 700,000 and has the chemical formula (C6H9NO)[n]. PVP is also known as poly[1 (2 oxo 1 pyrrolidinyl)ethylene]. PVP is nontoxic, highly hygroscopic and readily dissolves in water or organic solvents.

Polyvinyl alcohol (PVA) is a polymer prepared from polyvinyl acetates by replacement of the acetate groups with hydroxyl groups and has the chemical formula (CH2CHOH)[n]. Most polyvinyl alcohols are soluble in water.

Polyethylene glycol (PEG), also known as poly(oxyethylene) glycol, is a condensation polymer of ethylene oxide and water. PEG refers to a compound that includes repeating ethylene glycol units. The structure of PEG may be expressed as H—(O—CH2-CH2)n-OH. PEG is a hydrophilic compound that is biologically inert (i.e., non-immunogenic) and generally considered safe for administration to humans.

When PEG is linked to a particle, it provides advantageous properties, such as improved solubility, increased circulating life, stability, protection from proteolytic degradation, reduced cellular uptake by macrophages, and a lack of immunogenicity and antigenicity. PEG is also highly flexible and provides bio-conjugation and surface treatment of a particle without steric hindrance. PEG may be used for chemical modification of biologically active compounds, such as peptides, proteins, antibody fragments, aptamers, enzymes, and small molecules to tailor molecular properties of the compounds to particular applications. Moreover, PEG molecules may be functionalized by the chemical addition of various functional groups to the ends of the PEG molecule, for example, amine-reactive PEG (BS (PEG)n) or sulfhydryl-reactive PEG (BM (PEG)n).

In certain embodiments, the carrier is a biocompatible scaffold, such as a scaffold including polyethylene glycol (PEG). In a preferred embodiment, the carrier is a biocompatible scaffold that includes multiple subunits of covalently linked polyethylene glycol maleimide (PEG-MAL), for example, an 8-arm PEG-MAL scaffold. A PEG-containing scaffold may be selected because it is biocompatible, inexpensive, easily obtained commercially, has minimal uptake by the reticuloendothelial system (RES), and exhibits many advantageous behaviors. For example, PEG scaffolds inhibit cellular uptake of particles by numerous cell types, such as macrophages, which facilitates proper distribution to a specific tissues and increases residence time in the tissue.

An 8-arm PEG-MAL is a type of multi-arm PEG derivative that has maleimide groups at each terminal end of its eight arms, which are connected to a hexaglycerol core. The maleimide group selectively reacts with free thiol, SH, sulfhydryl, or mercapto group via Michael addition to form a stable carbon sulfur bond. Each arm of the 8-arm PEG-MAL scaffold may be conjugated to peptides, for example, via maleimide-thiol coupling or amide bonds.

The PEG-MAL scaffold may be of various sizes, for example, a 10 kDa scaffold, a 20 kDa scaffold, a 40 kDa scaffold, or a greater than 40 kDa scaffold. The hydrodynamic diameter of the PEG scaffold in phosphate buffered saline (PBS) may be determined by various methods known in the art, for example, by dynamic light scattering. Using such techniques, the hydrodynamic diameter of a 40 kDa PEG-MAL scaffold was measured to be approximately 8 nm. In preferred embodiments, a 40 kDa PEG-MAL scaffold is provided as the carrier when the activity sensor is administered subcutaneously because the activity sensor readily diffuses into systemic circulation but is not readily cleared by the reticuloendothelial system.

The size of the PEG-MAL scaffold affects the distribution and residence time of the activity sensor in the body because particles smaller than about 5 nm in diameter are efficiently cleared through renal filtration of the body, even without proteolytic cleavage. Further, particles larger than about 10 nm in diameter often drain into lymphatic vessels. In one example, where a 40 kDa 8-arm PEG-MAL scaffold was administered intravenously, the scaffold was not renally cleared into urine.

The reporter may be any reporter susceptible to an enzymatic activity, such that cleavage of the reporter indicates that enzymatic activity. The reporter is dependent on enzymes that are active in a specific disease state. For example, tumors are associated with a specific set of enzymes. For a tumor, the activity sensor may be designed with an enzyme susceptible site that matches that of the enzymes expressed by the tumor or other diseased tissue. Alternatively, the enzyme-specific site may be associated with enzymes that are ordinarily present but are absent in a particular disease state. In this example, a disease state would be associated with a lack of signal associated with the enzyme, or reduced levels of signal compared to a normal reference or prior measurement in a healthy subject.

In various embodiments, the reporter includes a naturally occurring molecule such as a peptide, nucleic acid, a small molecule, a volatile organic compound, an elemental mass tag, or a neoantigen. In other embodiments, the reporter includes a non-naturally occurring molecule such as D-amino acids, synthetic elements, or synthetic compounds. The reporter may be a mass-encoded reporter, for example, a reporter with a known and individually-identifiable mass, such as a polypeptide with a known mass or an isotope.

An enzyme may be any of the various proteins produced in living cells that accelerate or catalyze the metabolic processes of an organism. Enzymes act on substrates. The substrate binds to the enzyme at a location called the active site before the reaction catalyzed by the enzyme takes place. Generally, enzymes include but are not limited to proteases, glycosidases, lipases, heparinases, and phosphatases. Examples of enzymes that are associated with disease in a subject include but are not limited to MMP, MMP-2, MMP-7, MMP-9, kallikreins, cathepsins, seprase, glucose-6-phosphate dehydrogenase (G6PD), glucocerebrosidase, pyruvate kinase, tissue plasminogen activator (tPA), a disintegrin and metalloproteinase (ADAM), ADAM9, ADAM15, and matriptase. The detected enzymatic activity may be activity of any type of enzyme, for example, proteases, kinases, esterases, peptidases, amidases, oxidoreductases, transferases, hydrolases, lysases, isomerases, or ligases.

Examples of substrates for disease-associated enzymes include but are not limited to Interleukin 1 beta, IGFBP-3, TGF-beta, TNF, FASL, HB-EGF, FGFR1, Decorin, VEGF, EGF, IL2, IL6, PDGF, fibroblast growth factor (FGF), and tissue inhibitors of MMPs (TIMPs). Enzymes indicative of immune response can include, for example, tissue remodeling enzymes.

The tuning domains may include any suitable material that modifies a distribution or residence time of the activity sensor within a subject when the activity sensor is administered to the subject. For example, the tuning domains may include PEG, PVA, or PVP. In another example, the tuning domains may include a polypeptide, a peptide, a nucleic acid, a polysaccharide, volatile organic compound, hydrophobic chains, or a small molecule.

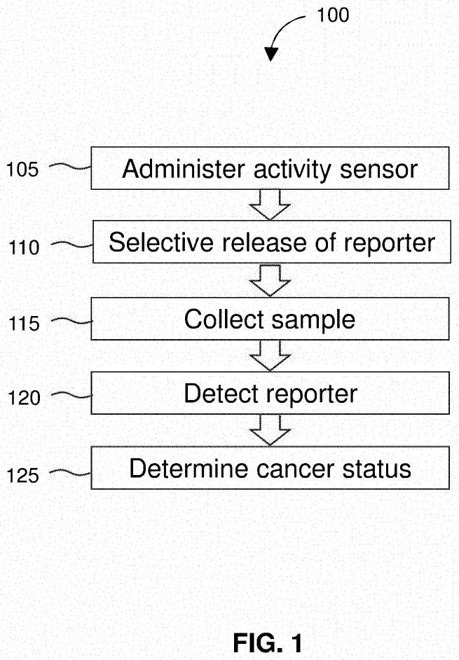

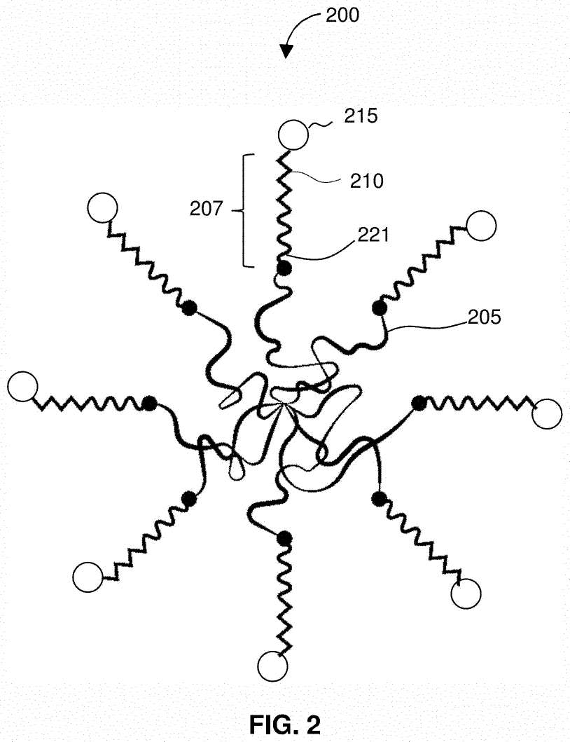

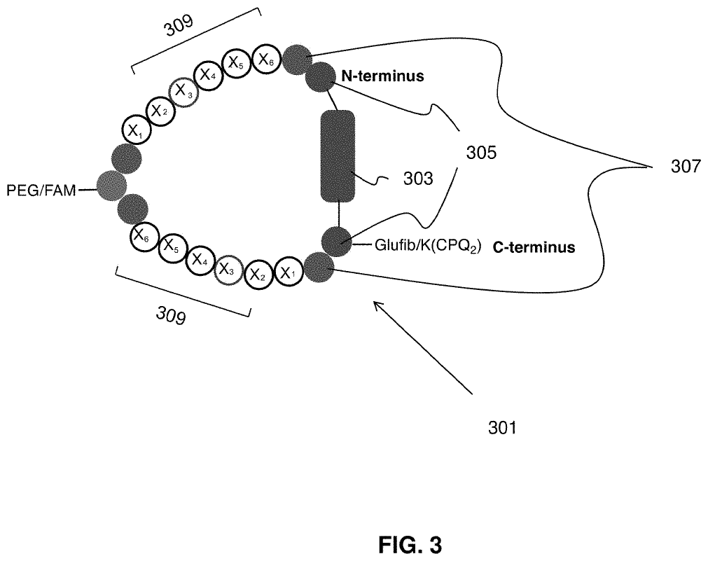

FIG. 1 diagrams steps of a method 100 for monitoring cancer progression. At step 105, an activity sensor is administered to a patient. The patient may be suspected of having cancer, known to have cancer (active or in remission), at risk of developing cancer, and/or undergoing treatment for cancer including immuno-oncological (I-O) therapies. The activity sensor includes a reporter linked by a cleavable linker to a carrier (e.g., as shown in FIGS. 2 and 3). The cleavable linker is sensitive to an enzyme for which the level is indicative of a characteristic in the tumor environment (e.g., enzymes upregulated in expanding tumors or tumors in regression, or enzymes indicative of active or inhibited immune responses). As discussed herein, depending on the enzyme activity the activity sensors are engineered to report on and the patient's disease and treatment status, information garnered from reporter levels in patient samples can be used to diagnose and/or stage the disease, monitor progression, predict responsiveness to a given therapy, and monitor therapeutic effectiveness including differentiating between anti-tumor immune response, general immune response, and tumor progression. Activity sensors can be administered by any suitable method. In preferred embodiments, the activity sensor is delivered intravenously or aerosolized and delivered to the lungs, for example, via a nebulizer. In other examples, the activity sensor may be administered to a subject transdermally, intradermally, intraarterially, intralesionally, intratumorally, intracranially, intraarticularly, intratumorally, intramuscularly, subcutaneously, orally, topically, locally, inhalation, injection, infusion, or by other method or any combination known in the art (see, for example, Remington's Pharmaceutical Sciences (1990), incorporated by reference).

At step 110, after administration of the activity sensor and localization of the activity sensor in the target tissue, the reporter is selectively released upon cleavage of the linker in the presence of the characteristic-indicative enzyme. Localization can be accomplished through the use of tuning domains including moieties preferentially concentrated in the target tissue (e.g., a specific tissue suspected of harboring cancer cells or concentrated in tumors generally). Upon release of the reporter, it can be cleared by the body into a fluid capable of non-invasive collection such as urine after transport to the blood stream and renal clearance.

At step 115, the sample, such as a urine sample, can be collected for analysis. At step 120, the sample can be analyzed and the presence and/or levels of the reporter in the sample can be detected. At step 125, the enzyme levels indicated by the presence of the reporter can be used to determine a characteristic associated with the observed differential expression. As noted above, depending on the enzyme sensitivity engineered into the activity sensors used, reporter levels can be used to monitor disease progression and I-O therapy response or to predict responsiveness to various treatments (e.g., determine hot or cold status of a tumor).

FIG. 2 shows an activity sensor 200 with carrier 205, reporters 207, and tuning domains 215. As illustrated, carrier 205 is a biocompatible scaffold that includes multiple subunits of covalently linked polyethylene glycol maleimide (PEG-MAL). Carrier 205 is an 8-arm PEG-MAL scaffold with a molecular weight between about 20 and 80 kDa. Reporter 207 is a polypeptide including a region susceptible to an identified protease. Activity of the identified protease to cleave the reporter indicates the disease. Reporter 207 includes a cleavable substrate 221 connected to detectable analyte 210. When a cleavage by the identified protease occurs upon cleavable substrate 221, detectable analyte 210 is released from activity sensor 200 and may pass out of the tissue, excreted from the body and detected.

In various embodiments, activity sensors may include cyclic peptides that are structurally resistant to non-specific proteolysis and degradation in the body. Cyclic peptides can include protease-specific substrates or pH-sensitive bonds that allow the otherwise non-reactive cyclic peptide to release a reactive reporter molecule in response to the presence of the enzymes discussed herein. Cyclic peptides can require cleavage at a plurality of cleavage sites to increase specificity. The plurality of sites can be specific for the same or different proteases. Polycyclic peptides can be used comprising 2, 3, 4, or more cyclic peptide structures with various combinations of enzymes or environmental conditions required to linearize or release the functional peptide or other molecule. Cyclic peptides can include depsipeptides wherein hydrolysis of one or more ester bonds releases the linearized peptide. Such embodiments can be used to tune the timing of peptide release in environments such as plasma.

FIG. 3 shows an exemplary cyclic peptide 301 having a protease-specific substrate 309 and a stable cyclization linker 303. The protease-specific substrate 309 may comprise any number of amino acids in any order. For example, X₁ may be glycine. X₂ may be serine. X₃ may be aspartic acid. X₄ may be phenylalanine. X₅ may be glutamic acid. X₆ may be isoleucine. The N-terminus and C-terminus, coupled to the cyclization linker 303 comprise cyclization residues 305. The peptide may be engineered to address considerations such as protease stability, steric hindrance around cleavage site, macrocycle structure, and rigidity/flexibility of peptide chain. The type and number of spacer residues 307 can be chosen to address and alter many of those properties by changing the spacing between the various functional sites of the cyclic peptide. The cyclization linker and the positioning and choice of cyclization residues can also impact the considerations discussed above. Tuning domains such as PEG and/or reporters such as FAM can be included in the cyclic peptide.

The biological sample may be any sample from a subject in which the reporter may be detected. For example, the sample may be a tissue sample (such as a blood sample, a hard tissue sample, a soft tissue sample, etc.), a urine sample, saliva sample, mucus sample, fecal sample, seminal fluid sample, or cerebrospinal fluid sample.

### Reporter Detection

Reporter molecules, released from activity sensors of the invention, may be detected by any suitable detection method able to detect the presence of quantity of molecules within the detectable analyte, directly or indirectly. For example, reporters may be detected via a ligand binding assay, which is a test that involves binding of the capture ligand to an affinity agent. Reporters may be directly detected, following capture, through optical density, radioactive emissions, or non-radiative energy transfers. Alternatively, reporters may be indirectly detected with antibody conjugates, affinity columns, streptavidin-biotin conjugates, PCR analysis, DNA microarray, or fluorescence analysis.

A ligand binding assay often involves a detection step, such as an ELISA, including fluorescent, colorimetric, bioluminescent and chemiluminescent ELISAs, a paper test strip or lateral flow assay, or a bead-based fluorescent assay.

In one example, a paper-based ELISA test may be used to detect the liberated reporter in urine. The paper-based ELISA may be created inexpensively, such as by reflowing wax deposited from a commercial solid ink printer to create an array of test spots on a single piece of paper. When the solid ink is heated to a liquid or semi-liquid state, the printed wax permeates the paper, creating hydrophobic barriers. The space between the hydrophobic barriers may then be used as individual reaction wells. The ELISA assay may be performed by drying the detection antibody on the individual reaction wells, constituting test spots on the paper, followed by blocking and washing steps. Urine from the urine sample taken from the subject may then be added to the test spots, then streptavidin alkaline phosphate (ALP) conjugate may be added to the test spots, as the detection antibody. Bound ALP may then be exposed to a color reacting agent, such as BCIP/NBT (5-bromo-4-chloro-3′-indolyphosphate p-toluidine salt/nitro-blue tetrazolium chloride), which causes a purple colored precipitate, indicating presence of the reporter.

In another example, volatile organic compounds may be detected by analysis platforms such as gas chromatography instrument, a breathalyzer, a mass spectrometer, or use of optical or acoustic sensors.

Gas chromatography may be used to detect compounds that can be vaporized without decomposition (e.g., volatile organic compounds). A gas chromatography instrument includes a mobile phase (or moving phase) that is a carrier gas, for example, an inert gas such as helium or an unreactive gas such as nitrogen, and a stationary phase that is a microscopic layer of liquid or polymer on an inert solid support, inside a piece of glass or metal tubing called a column. The column is coated with the stationary phase and the gaseous compounds analyzed interact with the walls of the column, causing them to elute at different times (i.e., have varying retention times in the column). Compounds may be distinguished by their retention times.

A modified breathalyzer instrument may also be used to detect volatile organic compounds. In a traditional breathalyzer that is used to detect an alcohol level in blood, a subject exhales into the instrument, and any ethanol present in the subject's breath is oxidized to acetic acid at the anode. At the cathode, atmospheric oxygen is reduced. The overall reaction is the oxidation of ethanol to acetic acid and water, which produces an electric current that may be detected and quantified by a microcontroller. A modified breathalyzer instrument exploiting other reactions may be used to detect various volatile organic compounds.

Mass spectrometry may be used to detect and distinguish reporters based on differences in mass. In mass spectrometry, a sample is ionized, for example by bombarding it with electrons. The sample may be solid, liquid, or gas. By ionizing the sample, some of the sample's molecules are broken into charged fragments. These ions may then be separated according to their mass-to-charge ratio. This is often performed by accelerating the ions and subjecting them to an electric or magnetic field, where ions having the same mass-to-charge ratio will undergo the same amount of deflection. When deflected, the ions may be detected by a mechanism capable of detecting charged particles, for example, an electron multiplier. The detected results may be displayed as a spectrum of the relative abundance of detected ions as a function of the mass-to-charge ratio. The molecules in the sample can then be identified by correlating known masses, such as the mass of an entire molecule to the identified masses or through a characteristic fragmentation pattern.

When the reporter includes a nucleic acid, the reporter may be detected by various sequencing methods known in the art, for example, traditional Sanger sequencing methods or by next-generation sequencing (NGS). NGS generally refers to non-Sanger-based high throughput nucleic acid sequencing technologies, in which many (i.e., thousands, millions, or billions) of nucleic acid strands can be sequenced in parallel. Examples of such NGS sequencing includes platforms produced by Illumina (e.g., HiSeq, MiSeq, NextSeq, MiniSeq, and iSeq 100), Pacific Biosciences (e.g., Sequel and RSII), and Ion Torrent by ThermoFisher (e.g., Ion S5, Ion Proton, Ion PGM, and Ion Chef systems). It is understood that any suitable NGS sequencing platform may be used for NGS to detect nucleic acid of the detectable analyte as described herein.

Analysis may be performed directly on the biological sample or the detectable analyte may be purified to some degree first. For example, a purification step may involve isolating the detectable analyte from other components in the biological sample. Purification may include methods such as affinity chromatography. The isolated or purified detectable analyte does not need to be 100% pure or even substantially pure prior to analysis.

Detecting the detectable analyte may provide a qualitative assessment (e.g., whether the detectable analyte is present or absent) or a quantitative assessment (e.g., the amount of the detectable analyte present) to indicate a comparative activity level of the enzymes. The quantitative value may be calculated by any means, such as, by determining the percent relative amount of each fraction present in the sample. Methods for making these types of calculations are known in the art.

The detectable analyte may be labeled. For example, a label may be added directly to a nucleic acid when the isolated detectable analyte is subjected to PCR. For example, a PCR reaction performed using labeled primers or labeled nucleotides will produce a labeled product. Labeled nucleotides, such as fluorescein-labeled CTP are commercially available. Methods for attaching labels to nucleic acids are well known to those of ordinary skill in the art and, in addition to the PCR method, include, for example, nick translation and end-labeling.

Labels suitable for use in the reporter include any type of label detectable by standard methods, including spectroscopic, photochemical, biochemical, electrical, optical, or chemical methods. The label may be a fluorescent label. A fluorescent label is a compound including at least one fluorophore. Commercially available fluorescent labels include, for example, fluorescein phosphoramidites, rhodamine, polymethadine dye derivative, phosphores, Texas red, green fluorescent protein, CY3, and CY5.

Other known techniques, such as chemiluminescence or colormetrics (enzymatic color reaction), can also be used to detect the reporter. Quencher compositions in which a “donor” fluorophore is joined to an “acceptor” chromophore by a short bridge that is the binding site for the enzyme may also be used. The signal of the donor fluorophore is quenched by the acceptor chromophore through a process believed to involve resonance energy transfer (RET), such as fluorescence resonance energy transfer (FRET). Cleavage of the peptide results in separation of the chromophore and fluorophore, removal of the quench, and generation of a subsequent signal measured from the donor fluorophore. Examples of FRET pairs include 5-Carboxyfluorescein (5-FAM) and CPQ2, FAM and DABCYL, Cy5 and QSY21, Cy3 and QSY7.

In various embodiments, the activity sensor may include ligands to aid it targeting particular tissues or organs. When administered to a subject, the activity sensor is trafficked in the body through various pathways depending on how it enters the body. For example, if activity sensor is administered intravenously, it will enter systemic circulation from the point of injection and may be passively trafficked through the body.

For the activity sensor to respond to enzymatic activity within a specific cell, at some point during its residence time in the body, the activity sensor must come into the presence of the enzyme and have an opportunity to be cleaved and linearized by the enzyme to release the linearized reporter or therapeutic molecule. From a targeting perspective, it is advantageous to provide the activity sensor with a means to target specific cells or a specific tissue type where such enzymes of interest may be present. To achieve this, ligands for receptors of the specific cell or specific tissue type may be provided as the tuning domains and linked to polypeptide.

Cell surface receptors are membrane-anchored proteins that bind ligands on the outside surface of the cell. In one example, the ligand may bind ligand-gated ion channels, which are ion channels that open in response to the binding of a ligand. The ligand-gated ion channel spans the cell's membrane and has a hydrophilic channel in the middle. In response to a ligand binding to the extracellular region of the channel, the protein's structure changes in such a way that certain particles or ions may pass through. By providing the activity sensor with tuning domains that include ligands for proteins present on the cell surface, the activity sensor has a greater opportunity to reach and enter specific cells to detect enzymatic activity within those cells.

By providing the activity sensor with tuning domains, distribution of the activity sensor may be modified because ligands may target the activity sensor to specific cells or specific tissues in a subject via binding of the ligand to cell surface proteins on the targeted cells. The ligands of tuning domains may be selected from a group including a small molecule; a peptide; an antibody; a fragment of an antibody; a nucleic acid; and an aptamer.

Once activity sensor reaches the specific tissue, ligands may also promote accumulation of the activity sensor in the specific tissue type. Accumulating the activity sensor in the specific tissue increases the residence time of the activity sensor and provides a greater opportunity for the activity sensor to be enzymatically cleaved by proteases in the tissue, if such proteases are present.

When the activity sensor is administered to a subject, it may be recognized as a foreign substance by the immune system and subjected to immune clearance, thereby never reaching the specific cells or specific tissue where the specific enzymatic activity can release the therapeutic compound or reporter molecule. Furthermore, generation of an immune response can defeat the purpose of immune-response-sensitive activity sensors. To inhibit immune detection, it is preferable to use a biocompatible carrier so that it does not elicit an immune response, for example, a biocompatible carrier may include one or more subunits of polyethylene glycol maleimide. Further, the molecular weight of the polyethylene glycol maleimide carrier may be modified to facilitate trafficking within the body and to prevent clearance of the activity sensor by the reticuloendothelial system. Through such modifications, the distribution and residence time of the activity sensor in the body or in specific tissues may be improved.

In various embodiments, the activity sensor may be engineered to promote diffusion across a cell membrane. As discussed above, cellular uptake of activity sensors has been well documented. See Gang. Hydrophobic chains may also be provided as tuning domains to facilitate diffusion of the activity sensor across a cell membrane may be linked to the activity sensor.

The tuning domains may include any suitable hydrophobic chains that facilitate diffusion, for example, fatty acid chains including neutral, saturated, (poly/mono) unsaturated fats and oils (monoglycerides, diglycerides, triglycerides), phospholipids, sterols (steroid alcohols), zoosterols (cholesterol), waxes, and fat-soluble vitamins (vitamins A, D, E, and K).

In some embodiments, the tuning domains include cell-penetrating peptides. Cell-penetrating peptides (CPPs) are short peptides that facilitate cellular intake/uptake of activity sensors of the disclosure. CPPs preferably have an amino acid composition that either contains a high relative abundance of positively charged amino acids such as lysine or arginine or has sequences that contain an alternating pattern of polar/charged amino acids and non-polar, hydrophobic amino acids. See Milletti, 2012, Cell-penetrating peptides: classes, origin, and current landscape, Drug Discov Today 17:850-860, incorporated by reference. Suitable CPPs include those known in the literature as Tat, R6, R8, R9, Penetratin, pVEc, RRL helix, Shuffle, and Penetramax. See Kristensen, 2016, Cell-penetrating peptides as tools to enhance non-injectable delivery of biopharmaceuticals, Tissue Barriers 4(2):e1178369, incorporated by reference.

In certain embodiments, an activity sensor may include a biocompatible polymer as a tuning domain to shield the activity sensor from immune detection or inhibit cellular uptake of the activity sensor by macrophages.

When a foreign substance is recognized as an antigen, an antibody response may be triggered by the immune system. Generally, antibodies will then attach to the foreign substance, forming antigen-antibody complexes, which are then ingested by macrophages and other phagocytic cells to clear those foreign substances from the body. As such, when an activity sensor enters the body, it may be recognized as an antigen and subjected to immune clearance, preventing the activity sensor from reaching a specific tissue to detect enzymatic activity. To inhibit immune detection of the activity sensor, for example, PEG tuning domains may be linked to the activity sensor. PEG acts as a shield, inhibiting recognition of the activity sensor as a foreign substance by the immune system. By inhibiting immune detection, the tuning domains improve the residence time of the activity sensor in the body or in a specific tissue.

Enzymes have a high specificity for specific substrates by binding pockets with complementary shape, charge and hydrophilic/hydrophobic characteristic of the substrates. As such, enzymes can distinguish between very similar substrate molecules to be chemoselective (i.e., preferring an outcome of a chemical reaction over an alternative reaction), regioselective (i.e., preferring one direction of chemical bond making or breaking over all other possible directions), and stereospecific (i.e., only reacting on one or a subset of stereoisomers).

Steric effects are nonbonding interactions that influence the shape (i.e., conformation) and reactivity of ions and molecules, which results in steric hindrance. Steric hindrance is the slowing of chemical reactions due to steric bulk, affecting intermolecular reactions. Various groups of a molecule may be modified to control the steric hindrance among the groups, for example to control selectivity, such as for inhibiting undesired side-reactions. By providing the activity sensor with tuning domains such as spacer residues between the carrier and the cleavage site and/or any bioconjugation residue, steric hindrance among components of activity sensor may be minimized to increase accessibility of the cleavage site to specific proteases. Alternatively, steric hindrance can be used as described above to prevent access to the cleavage site until an unstable cyclization linker (e.g., an ester bond of a cyclic depsipeptide) has degraded. Such unstable cyclization linkers can be other known chemical moieties that hydrolyze in defined conditions (e.g., pH or presence of a certain analyte) which may be selected to respond to specific characteristics of a target environment.

In various embodiments, activity sensors may include D-amino acids aside from the target cleavage site to further prevent non-specific protease activity. Other non-natural amino acids may be incorporated into the peptides, including synthetic non-native amino acids, substituted amino acids, or one or more D-amino acids.

In some embodiments, tuning domains may include synthetic polymers such as polymers of lactic acid and glycolic acid, polyanhydrides, polyurethanes, and natural polymers such as alginate and other polysaccharides including dextran and cellulose, collagen, albumin and other hydrophilic proteins, zein and other prolamines and hydrophobic proteins, copolymers and mixtures thereof.

One of skill in the art would know what peptide segments to include as protease cleavage sites in an activity sensor of the disclosure. One can use an online tool or publication to identify cleavage sites. For example, cleavage sites are predicted in the online database PROSPER, described in Song, 2012, PROSPER: An integrated feature-based tool for predicting protease substrate cleavage sites, PLoSOne 7(11):e50300, incorporated by reference. Any of the compositions, structures, methods or activity sensors discussed herein may include, for example, any suitable cleavage site, as well as any further arbitrary polypeptide segment to obtain any desired molecular weight. To prevent off-target cleavage, one or any number of amino acids outside of the cleavage site may be in a mixture of the D and/or the L form in any quantity.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of detecting or monitoring cancer progression comprising:
administering to a patient suspected of having cancer an activity sensor comprising a carrier linked to a reporter molecule by a cleavable linker containing a cleavage site of a cancer-related enzyme;
collecting a sample from the patient;
analyzing the sample to detect the presence or lack of the reporter wherein the presence or lack of the reporter is indicative of cancer status for the patient.

2. The method of claim 1, wherein the cancer-related enzyme is an immunological enzyme and presence of the reporter is indicative of an anti-tumor immune response.

3. The method of claim 2, wherein the presence of the reporter is indicative of therapeutic effect of an immuno-oncology treatment.

4. The method of claim 3, wherein said therapeutic effect is a measure of resistance to a pharmaceutical treatment.

5. The method of claim 1, wherein said reporter is a near-IR dye, a nucleic acid barcode, or a protein biomarker.

6. The method of claim 1, wherein said carrier is a synthetic carrier.

7. The method of claim 1, wherein said carrier is a naturally-occurring carrier.

8. The method of claim 1, wherein said administering step is selected from the group consisting of intravenous, oral, transdermal, inhalation, and intracerebral.

9. The method of claim 3, wherein the activity sensor further comprises a tuning domain operable to localize the activity sensor in a target tumor.

10. The method of claim 1, wherein the cancer-related enzyme is an enzyme differentially expressed during tumor growth and the presence of the reporter is indicative of tumor growth.

11. The method of claim 1, wherein the cancer-related enzyme is an enzyme differentially expressed during cell death, and wherein the presence of the reporter is indicative of a therapeutic response to the anti-cancer therapy.

12. The method of claim 1, wherein the patient has not undergone immuno-oncology treatment and the presence of the reporter is indicative of a predicted therapeutic response to a checkpoint inhibitor therapy.

13. The method of claim 1, further comprising stratifying the patient in a clinical trial based on the detection of the reporter in the sample.

14. The method of claim 1, wherein the analyzing step further comprises quantifying a level of the reporter in the sample, the method further comprising:
periodically repeating the administering, collecting, and analyzing steps to prepare a chronological series of reporter levels, and
determining a velocity of the characteristic indicative of cancer progression in the patient.

15. The method of claim 1, wherein the immunological enzyme is selected from the group consisting of a caspase and a serine protease.

16. An activity sensor for monitoring cancer progression, the activity sensor comprising:
a carrier comprising one or more molecular subunits;
a plurality of detectable reporters, each linked to the carrier by a cleavable linker containing the cleavage site of a cancer-related enzyme; and
a tuning domain operable to localize the activity sensor in a target tumor, wherein the activity sensor reports activity of one or more cancer-related enzymes by releasing the reporters upon cleavage by the one or more cancer-related enzymes.

17. The activity sensor of claim 16, wherein the cancer-related enzyme is an immunological enzyme.

18. The activity sensor of claim 17, wherein the immunological enzyme is selected from the group consisting of a caspase and a serine protease.

19. The activity sensor of claim 16, wherein the tuning domain comprises ligands for receptors of the target tumor.

20. The activity sensor of claim 16, wherein carrier comprises a polyethylene glycol (PEG) scaffold of covalently linked PEG subunits.
